(19) United States
(12) Patent Application Publication
(10) Pub. No.: US 2009/0270689 A1
(43) Pub. Date: Oct. 29, 2009

(54) MONITORING SYSTEM

(75) Inventor: Beaufort Galland, Victoria (AU)

Correspondence Address: SHERIDAN ROSS PC, 1560 BROADWAY, SUITE 1200, DENVER, CO, 80202, US

(73) Assignee: CBB INTERNATIONAL PTY LTD, South Melbourne, Victoria (AU)

(21) Appl. No.: 12/303,111

(22) PCT Filed: Jun. 1, 2007
(86) PCT No.: PCT/AU2007/000762

§ 371 Date: Jul. 1, 2009

(30) Foreign Application Priority Data
Jun. 2, 2006 (AU) 2006903012

(51) Int. Cl. A61B 5/00

(52) U.S. Cl. 600/300

(57) ABSTRACT

A system (10) for monitoring physiological parameters of a user. The system (10) includes a sensor unit. The sensor unit includes a sensor for sensing physiological information for at least one physiological parameter of the user; and a transmitter for wirelessly transmitting a first signal containing the sensed information. The system (10) also includes a portable unit (12). The portable unit (12) includes a portable unit controller; a memory for storing preprogrammed information; a receiver for receiving, under the control of the controller, the first signal into memory as sensed physiological data; and a transmitter for wirelessly transmitting, under the control of the controller, a second signal containing the preprogrammed information. The system (10) further includes a user output device (14) including a second receiver for receiving the second signal and a converter for converting the second signal into an output that is audible to the user.

The present invention generally relates to a monitoring system for a user. More particularly, the invention relates to a system having the ability to monitor a range of parameters of a user, including physiological parameters and will herein be generally described in that context. It is to be appreciated, however, that the invention may be adapted for other applications.

The monitoring of physiological parameters, such as in the context of an exercise or sports training aide, has become increasingly popular by both elite athletes and the general public. One example product is that of a heart rate monitor, which is capable of providing the user with information relating to their heart rate. Heart rate monitors often include a sensor/transmitter unit for sensing and wirelessly transmitting a data signal relating to the user's heart rate, and a receiver for wirelessly receiving the signal from the transmitter. The sensor and transmitter can be in the form of a chest band unit worn by the user, while the receiver can be in the form of a wrist-mounted unit including a readout display for displaying details of the user's heart rate.

A variety of other potentially useful data can also be sensed and displayed by these types of monitors. For example, a bike sensor(s) may be incorporated into this type of product, with the monitor optionally being mounted on the user's bike for displaying the user's cycling speed, trip distance and cadence.

The output available from these types of existing monitors is often limited to the information provided visually by way of the readout display. This requires the user to actually look at the display, which may be difficult or dangerous during exercise and can be even more difficult in some conditions, such as in poor light. While some monitors provide an audible “beep” or alarm indicating, for example, a time interval or a heart rate level reached by the user, such an audible output

can provide insufficient information alone, thus still requiring the user to look at the readout display to determine the cause of the beep.

The above discussion is provided generally in the context of providing a monitor for use during exercise. However, it is to be appreciated that the invention has far broader application including, but not limited to, a wide range of outdoor activities, medical monitoring uses and other uses by individuals in the course of their daily lives.

It would be desirable to provide an alternative and/or improved monitor that potentially addresses the drawbacks of existing monitors as referred to above.

According to one broad aspect of the present invention there is provided a system for monitoring physiological parameters of a user. The system includes:
(a) a sensor unit including:
   a sensor for sensing physiological information for at least one physiological parameter of the user; and
   a transmitter for wirelessly transmitting a first signal containing the sensed information;
(b) a portable unit including:
   a portable unit controller
   a memory for storing preprogrammed information;
   a receiver for receiving, under the control of the controller, the first signal into memory as sensed physiological data;
   a transmitter for wirelessly transmitting, under the control of the controller, a second signal containing the preprogrammed information;
(c) a user output device including a second receiver for receiving the second signal and a converter for converting the second signal into an output that is audible to the user; and wherein
   the sensor unit further includes a sensor for sensing environmental information for at least one environmental parameter to which the user is exposed.

The system may include a base unit, the base unit including a docking facility for docking the portable unit thereto.

In a particularly preferred form, the preprogrammed information includes audio information. The preprogrammed information may be provided in one or more data files such as, for example, one or more mp3, mp4 and m4a files or other current audio format. The data files may include music.

In a further embodiment, the music may be overlaid with exercise information and/or instructions informing and/or instructing the user of facts or specific tasks. This may include providing the user with specific exercise information or tasks at certain points during a music track. For example, the user or the user's coach or personal trainer could use the system as a training or fitness tool by providing audible coaching or workout instructions to the user which are overlaid onto, within or between music tracks for the benefit of the user.

The second signal transmitted by the transmitter of the portable unit may include both the preprogrammed information and information related to the sensed physiological data. The provision of the second signal transmitting information related to the sensed physiological data could advantageously provide the user with audio information relating to the physiological data, rather than the need to refer to a display, as is necessary when using a number of existing systems. For example, the second signal may transmit information on the user's current heart rate, body and/or skin temperature to, in turn, provide an audio output for the user on their current exercise work rate.

In one preferred form, the transmitter of the sensor unit and the receiver of the portable unit are connected by a Bluetooth connection and the first signal is a Bluetooth signal.

Further, the transmitter of the portable unit and the receiver of the user output device are preferably connected by a Bluetooth connection and the second signal is a Bluetooth signal.

Other, wireless connections are, of course, also contemplated.

Preferably, the portable unit is configured to be worn or carried by the user. The portable unit may be incorporated or integrated into an article of clothing, headwear or footwear. Alternatively, the portable unit may simply be carried in the hand or garment pocket of the user. In yet another arrangement, and in the context of the user being a cyclist, the portable unit could be mounted on a bicycle. In each of these arrangements it is advantageous for the portable unit to be relatively small and easily carried, so as not to inconvenience or obstruct the user during exercise. Further, it is advantageous that the portable unit be housed in a water-proof or at least water-resistant housing to protect it from moisture, since the unit is likely to be exposed to perspiration, water and other moisture sources during exercise.

In one particularly preferred form, the sensor of the sensor unit is integrated into the fabric or material of an article to be worn by the user. For example, the sensor may be provided in a thread-like form and weaved into the fabric or material of the article. The article may be any one or more of an article of clothing, headwear or footwear. The transmitter of the sensor unit may likewise be integrated into the article.

The sensor unit has, so far been described in the context of having a single sensor. However, the system may include a plurality of sensors for sensing a variety of different physiological parameters. For example, one sensor may be integrated into a garment to be worn by the user for sensing the user's heart rate and a second sensor may be incorporated into the/another garment for measuring the user's body (i.e., skin) temperature.

The sensor unit may include one or more sensors for measuring the acceleration of an individual. Such information may be of benefit for later analysis of the user's impact loading, particularly when used to record parameters during, for example, ball and other collision/impact sports.

Similarly, the sensor unit may include one or more sensors for measuring the inclination of an individual. This information may provide useful information for later analysis, and could, for example, measure a user's cornering inclination when cycling.

It is envisaged that, in one form, the controller includes a processing unit and software for controlling the content and transmission of the second signal from the transmitter of the portable unit to the receiver of the output device.

Preferably, the monitoring system also includes a base unit. The base unit may include a base unit transmitter for wirelessly transmitting a third signal containing the preprogrammed information to a second receiver of the portable unit for storage in the memory of the portable unit. The base unit may also include a base unit receiver for wirelessly receiving a fourth signal containing the sensed physiological data from the memory of the portable unit by way of a second transmitter of the portable unit.

The transmitter of the base unit and the second receiver of the portable unit are, in a preferred form, connected by a Bluetooth connection and the third signal is a Bluetooth signal. Likewise, the receiver of the base unit and the second transmitter of the portable unit are, in a preferred form, connected by a Bluetooth connection and the fourth signal is a Bluetooth signal.

The receiver and second receiver of the portable unit may be integrated. The transmitter and second transmitter of the portable unit may also be integrated.

It is envisaged that the monitoring system may also monitor environmental information to which the user is exposed. To this end the sensor unit may include a sensor for sensing environmental information for at least one environmental parameter to which the user is exposed. Preferred environmental parameters include at least one of the user's location, altitude, humidity, barometric pressure and ambient temperature.

Each of the sensors for sensing environmental information may adopt any suitable form. They may, for example be integrated into or connectable to an article(s) to be worn by the user and wirelessly connected to the portable unit. Alternatively they may be integrated into the portable unit, thereby obviating the need for a wireless connection between the sensors and the portable unit.

The user output device may adopt any suitable form. In one form, the user output device includes an audio speaker for providing the audio output. The output device may include a pair of speakers and the pair of speakers may be in the form of a pair of wireless earphones and/or a wireless headset and/or a pair of wireless headphones. The user output device may also include a wireless miniature receiver and/or communication earpiece to communicate with another like device and/or telephone. The communication earpiece may include both a speaker and a microphone allowing, for example, communication between the system user and another person. Hereinafter, unless otherwise stated, reference to earphones is understood to include any one or more of a headset, headphones, earphones, miniature receiver and communication earpiece. Each of the above user output device arrangements provide the advantage of being potentially less likely to become dislodged from the user's outer ear, when compared to earphones having a wired connection between the unit transmitter and earphones. This is because wired connections tend to flail about during exercise, which can lead to the earphones of this type being dislodged from the user's outer ear.

The earphones may include a microphone for voice recordal of the user. A suitable recordal storage device may be provided on the earphone set or on, for example, the portable unit.

In one preferred form, at least one of the wireless earphones includes an audio output controller for controlling the audible output. The output controller may include any one or more of a volume controller, controllers for starting and stopping the output, and controllers for scrolling forwardly or backwardly through the output. In the situation where the preprogrammed information is provided in more than one data file then the output controller may include a controller for scrolling forwardly or backwardly through the data files.

Preferably, the earphones include a rechargeable battery or replacement battery, such as in the form of a watch-type battery or batteries.

The earphones may be integrated or otherwise connected to, for example, a bicycle helmet or other protective helmet or headwear generally.

The system may also include a camera for recording images during use of the system by the user. The camera may be configured for recording video and/or still images.

It is envisaged that, in one form, the portable unit includes a rechargeable battery and the base unit includes a battery recharger for recharging the battery. In such an arrangement, the battery charger may be an inductive battery charger for recharging the battery of the portable unit when the portable unit is “docked” upon or otherwise in contact with the base unit. This arrangement desirably provides a particularly user-friendly, safe and simple arrangement for users of the system to easily and safely recharge the battery of the portable unit.

The base unit may also be configured for recharging rechargeable batteries provided in/with the earphones and/or camera.

The portable unit may be integrated into the earphones, thereby obviating the portable unit as a separate entity.

The base unit is preferably connectable to mains power and includes a port, such as a USB port, for connecting to and uploading the preprogrammed information from, for example, a computer. The USB port may also be used to download the sensed physiological data to a computer to enable the recorded data to be analysed. Computer software for analyzing the data is preferably included with the system.

It is to be appreciated that the system may also include a portable monitor, such as a wrist-mounted monitor having a display so as to provide the user with real time information about the physiological data, environmental data and data being received by the earphones. The portable monitor may include a GPS unit and an altimeter.

According to another aspect of the present invention, there is provided a system for monitoring physiological parameters of a user, the system including:
(a) a sensor unit including:
   a sensor for sensing physiological information for at least one physiological parameter of the user; and
   a transmitter for wirelessly transmitting a first signal containing the sensed information; and
(b) a portable unit including:
   a portable unit controller
   a memory for storing preprogrammed information;
   a receiver for receiving, under the control of the controller, the first signal into memory as sensed physiological data;
   a transmitter for wirelessly transmitting, under the control of the controller, a second signal containing the preprogrammed information; and wherein
   the sensor unit further includes a sensor for sensing environmental information for at least one environmental parameter to which the user is exposed.

It will be convenient to hereinafter describe preferred embodiments of the invention with reference to the accompanying drawings. The particularity of the drawings is to be understood as not limiting the preceding broad description of the invention.

In the drawings:

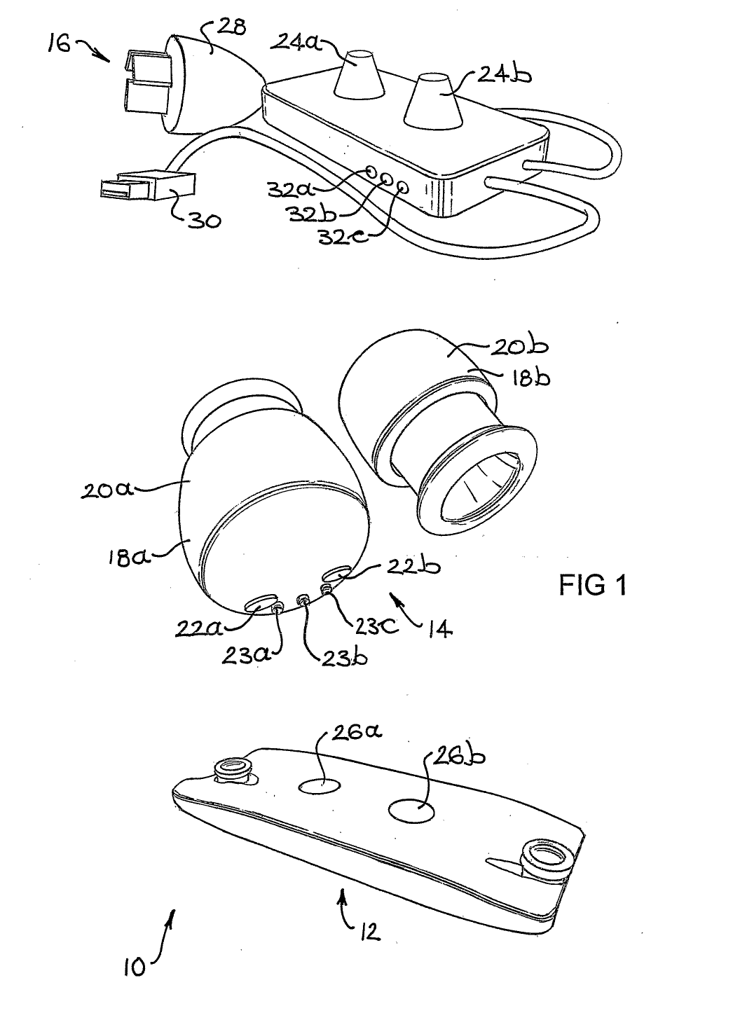

FIG. 1 is a perspective view of various elements of a monitoring system according to one aspect of the present invention.

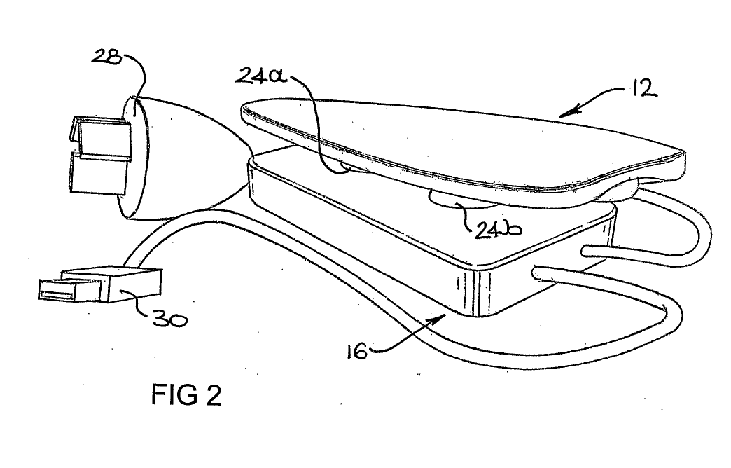

FIG. 2 is a perspective view of the portable unit illustrated in FIG. 1 “docked” on the base unit illustrated in FIG. 1.

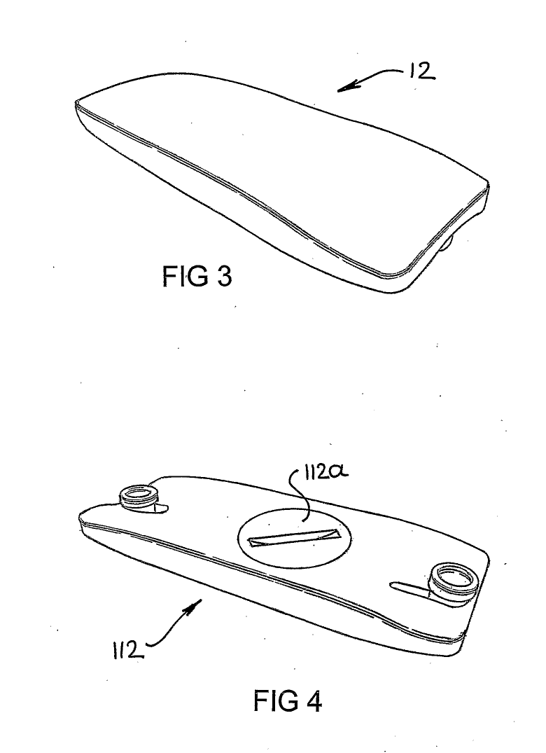

FIG. 3 is a perspective view of the upper side of the portable unit illustrated in FIG. 1.

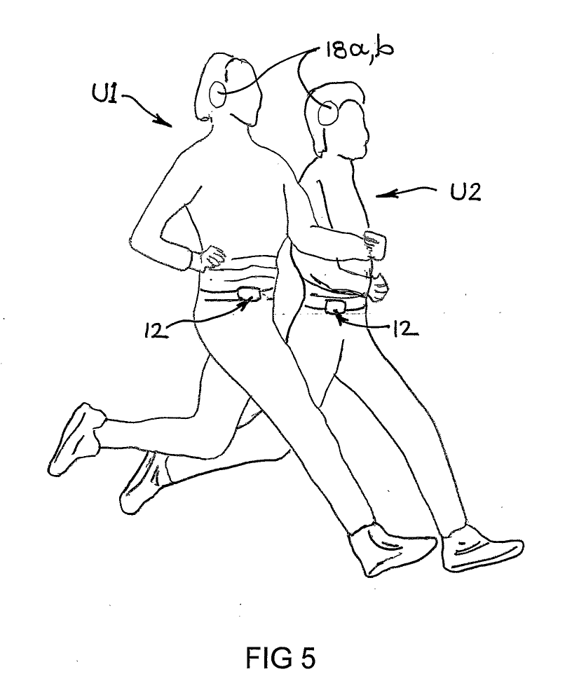

FIG. 4 is a perspective under side view of a portable unit according to another aspect of the present invention.

FIG. 5 is an illustration indicating how the monitoring system of FIG. 1 may be used.

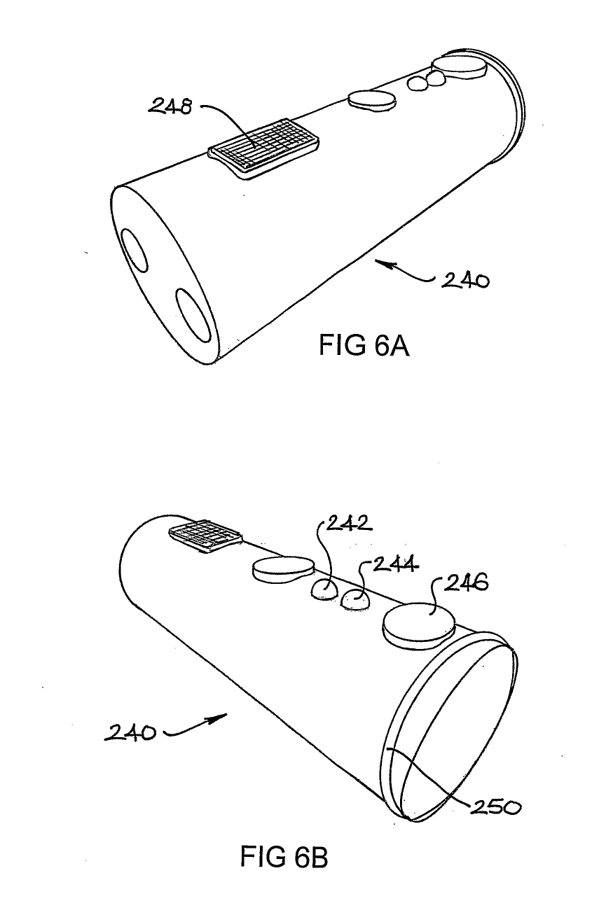

FIGS. 6a and 6b are perspective views of a camera of a monitoring system according to another aspect of the present invention.

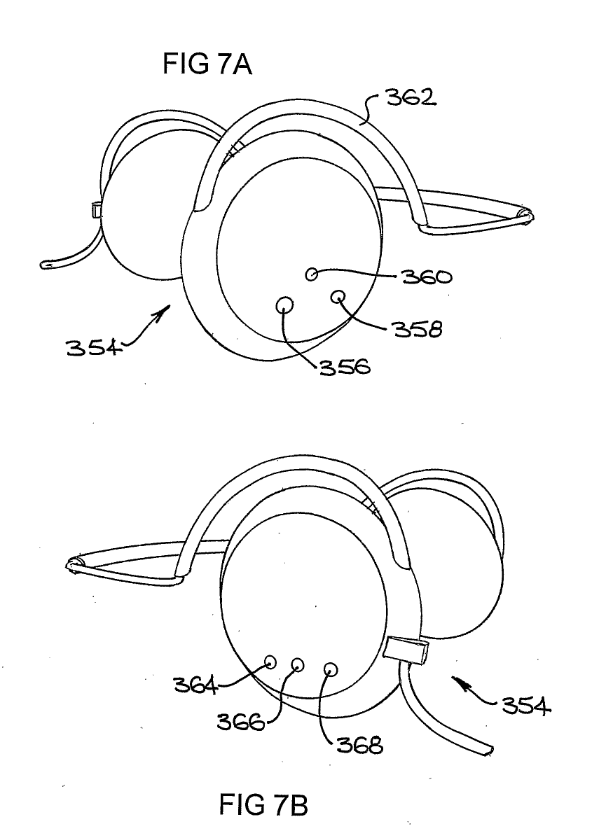

FIGS. 7a and 7b are perspective views of a headset of a monitoring system according to another aspect of the present invention.

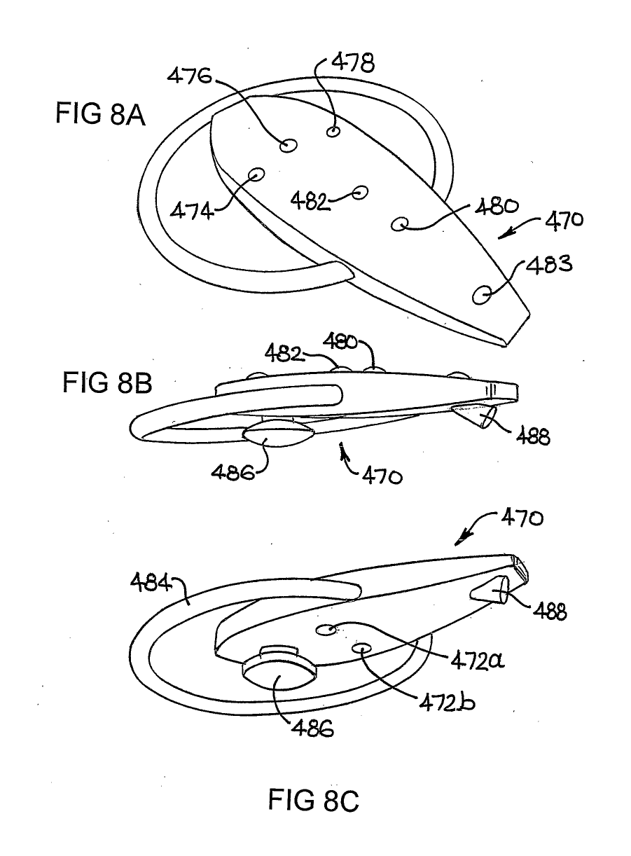

FIGS. 8a, 8b and 8c are perspective views of a receiver/communication earpiece of a monitoring system according to another aspect of the present invention.

Referring to FIGS. 1 to 3 and 5, there is illustrated a monitoring system 10. The system 10 includes a sensor unit (not illustrated), a portable unit 12, a user output device 14 and a base unit 16. The monitoring system 10 enables a user U1,U2 to record selected physiological information, including their skin temperature and heart rate. Two user's U1,U2 are illustrated in FIG. 5, each using a separate and individual monitoring system 10.

The sensor unit includes two sensors (not illustrated) for sensing physiological information for two physiological parameters of the user. Both sensors are integrated into a garment of clothing worn by the user U1,U2. One sensor is provided for sensing the user's heart rate and the second sensor is provided for sensing the user's body/skin temperature.

The sensor unit also includes a transmitter for wirelessly transmitting a first signal containing the sensed information relating to the user's heart rate and skin temperature.

The system 10 includes a portable unit 12. The portable unit 12 is configured to be worn or carried by the user. The portable unit 12 is provided in a small, slim, anatomical, waterproof casing, which is connected to, integrated or otherwise incorporated into an article of clothing, headwear or footwear worn by the user. Alternatively, the portable unit 12 may simply be carried in the pocket of a garment worn by the user U1,U2 or by hand. In yet another arrangement, and in the context of the user being a cyclist, the portable unit could be mounted on a bicycle ridden by the user.

The portable unit 12 contains a controller, and a memory for storing preprogrammed information. The portable unit 12 also contains a receiver for receiving, under the control of the controller, the first signal into memory as sensed physiological data relating to the user's heart rate and body/skin temperature; and a transmitter for wirelessly transmitting, under the control of the controller, a second signal containing preprogrammed information stored in the memory.

As illustrated, the system 10 further includes a user output device 14 in the form of a pair of wireless earphones 18a,b. Wireless earphones provide the advantage of being potentially less likely to become dislodged from the user's outer ear, when compared to earphones having a wired connection between the portable unit transmitter and earphones. This is because wired connections tend to flail about during exercise, which can lead to the earphones of this type being dislodged from the user's outer ear.

Within the earphone housings 20a,b there is provided a second receiver for receiving the second signal from the portable unit 12 and a converter for converting the second signal into an output that is audible to the user via the earphones 18a,b.

One of the wireless earphones 18a includes an audio output controller in the form of a pair of buttons 22a,b for controlling the audible output. The buttons 22a,b may be provided for any one or more of volume control buttons, output start and stop buttons, and scroll buttons for scrolling forwardly or backwardly through the output. In the situation where the preprogrammed information is provided in more than one data file then the buttons 22a,b may include a controller for scrolling forwardly or backwardly through the data files. The earphone 18a also includes mode indicators in the form of an array of LED lights 23a,b,c, which may be used to indicate, for example, that a signal is being received by the earphones 18a,b.

The earphones 18a,b each include a rechargeable lithium ion battery, a water proof housing 20a,b and a water proof speaker.

The memory of the portable unit 12 stores preprogrammed information in the form of one or more data files such as, for example, one or more mp3 music files. The one or more data files will be assumed to be music files for the remainder of this description.

The music provided in the music files may be overlaid with exercise information and/or instructions informing and/or instructing the user of facts or specific tasks. This may include providing the user with specific exercise information or tasks at certain points during a music track. As previously stated, the user or the user's coach or personal trainer could use the system as a training or fitness tool by providing audible coaching or workout instructions to the user which are overlaid onto, within or between music tracks for the benefit of the user. The audio output could be used to, for example, instruct the user to increase or decrease their physical output at any point in time in accordance with a pre-determined training program or workout.

As further previously indicated, the second signal transmitted by the transmitter of the portable unit 12 can include both the preprogrammed information and information related to the sensed physiological data. The provision of the second signal transmitting information related to the sensed physiological data could advantageously provide the user U1,U2 (via control of the portable unit controller) with audio information relating to the physiological data, rather than the need to refer to a display, as is necessary when using a number of existing systems. For example, the second signal may transmit information on the user's current heart rate and/or body/skin temperature to, in turn, provide an audio output via the earphones 18a,b for the user on their current physiological data and exercise work rate. The audio output could also, for example, provide the user with a verbal warning that their heart rate is above a predetermined limit and that they should reduce their physical output accordingly.

The transmitter of the sensor unit and the receiver of the portable unit 12 are connected by a Bluetooth connection and the first signal is a Bluetooth signal.

Further, the transmitter of the portable unit 12 and the receiver of the user output device 14 are connected by a Bluetooth connection and the second signal is a Bluetooth signal.

The sensor of the sensor unit is integrated into the fabric or material of an article worn by the user U1,U2. More particularly, each sensor of the sensor unit is provided in a thread-like form and weaved into the fabric or material of the article. The article may be any one or more of an article of clothing, headwear or footwear and is preferably the same article of clothing to which the portable unit 12 is mounted.

The controller of the portable unit 12 includes a micro chip processing unit and software for controlling the content and transmission of the second signal from the transmitter of the portable unit 12 to the receiver of the earphones 18a,b. The portable unit may include a multi media card.

The base unit 16 includes a base unit transmitter for wirelessly transmitting a third signal containing the preprogrammed information to a second receiver of the portable unit 12 for storage in the memory of the portable unit 12. The base unit 16 also includes a base unit receiver for wirelessly receiving a fourth signal containing the sensed physiological (i.e., heart rate, body and skin temperature) data from the memory of the portable unit 12 by way of a second transmitter in the portable unit 12.

The transmitter of the base unit 16 and the second receiver of the portable unit 12 are connected by a Bluetooth connection and the third signal is a Bluetooth signal. Likewise, the receiver of the base unit and the second transmitter of the portable unit are connected by a Bluetooth connection and the fourth signal is a Bluetooth signal.

In the illustrated embodiment, the receiver and second receiver of the portable unit 12 are integrated. The transmitter and second transmitter of the portable unit 12 are also integrated.

The monitoring system 10 may be configured for also monitoring environmental information to which the user is exposed, as well as their physiological information. To this end, the sensor unit may include a sensor for sensing environmental information for at least one environmental parameter to which the user is exposed. Environmental parameters contemplated include at least one of the user's location, altitude, humidity, barometric pressure and ambient air temperature.

Each of the sensors for sensing environmental information may adopt any suitable form. They may, for example be integrated into or connectable to an article(s) to be worn by the user and wirelessly connected to the portable unit 12. Alternatively they may be integrated into the portable unit 12, thereby obviating the need for a wireless connection between the sensors and the portable unit.

The environmental information may be recorded in the memory of the portable unit 12 for later analysis and/or may be sent via a signal to the earphones 18a,b to provide the user with an audio message as to their current location, altitude, or the current humidity, barometric pressure or ambient air temperature.

The portable unit 12 includes a rechargeable battery and the base unit 16 includes a battery recharger for recharging the battery. The base unit battery may be a rechargeable micro lithium ion or compatible battery. The battery charger is an inductive battery charger for recharging the battery of the portable unit 12 when the portable unit 12 is “docked” upon the base unit 16, as illustrated in FIG. 2. In this respect, the base unit 16 includes a pair of male members 24a,b for receipt within a pair of female members 26a,b provided on the portable unit 12. This arrangement desirably provides a particularly user-friendly and simple arrangement for users of the system 10 to easily and safely recharge the battery of the portable unit 12.

The base unit 16 (and battery charger) is connectable to AC mains power via a plug 28. The base unit 16 also includes a USB port 30, for connecting to and uploading the preprogrammed information from, for example, a computer. The USB port 30 is also used to download the sensed physiological and environmental data from the memory of the portable unit 12 to a computer to enable the recorded data to be analyzed. Computer software is included with the system for analyzing this data.

The base unit 16 includes indicator lights 32a,b,c (illustrated in FIG. 1 but not in FIG. 2) for indicating the various operating modes of the base unit (for example a power ON/OFF indicator, a recharging indicator, Bluetooth connection indicator and a data downloading/uploading indicator)

In use, the user U1,U2 connects the base unit 16 to a mains power supply via plug 28 and connects the base unit to a computer via USB port 30.

Following this, data such as music files may be wirelessly uploaded (using suitable software purchased with the system 10 and previously downloaded onto the computer) from the computer, via the base unit 16 to the portable unit 12.

Once this uploading of data has been completed the portable unit 12 may be connected to, integrated or otherwise incorporated into an article of clothing, headwear or footwear worn by the user (if is not already). Alternatively, the portable unit 12 may simply be carried in the pocket of a garment worn by the user U1,U2 or by hand or, in the context of the user being a cyclist, the portable unit could be mounted on a bicycle ridden by the user.

The user U1,U2 then dons the article of clothing, headwear, footwear carrying the sensors for recording both physiological and environmental data, and places the earphones 18a,b in his/her outer ears. The system is then switched ON by a suitably located button, following which sensed data received by the sensors begins being wirelessly transmitted by the sensors to the portable unit 12 under the control of the controller for later downloading to the computer for analysis.

At the same time as physiological and environmental data is being recorded in the memory of the portable unit 12, the preprogrammed data stored in the memory of the portable unit can be transmitted wirelessly (under the control of the portable unit controller) to the earphones 18a,b for the listening pleasure of the user U1,U2. This preprogrammed data may simply include music or may also include, for example, specific instructions or advice for the user to take in while exercising.

Optionally, the portable unit controller may be able to control and direct certain physiological and/or environmental information to the user U1,U2 via an audio output through the earphones 18a,b, thereby providing the user with a potentially real time audio output of their current activities and surroundings. For example, an audio output may be provided advising the user of any one of more of their current heart rate, their skin temperature, ambient air temperature and their current altitude.

Although not illustrated, system 10 may also include a portable monitor, such as a wrist-mounted monitor having a display so as to provide the user with real time information about the physiological data, environmental data and data being received by the earphones 18a,b.

FIG. 4 illustrates a second embodiment of a portable unit 112. The portable unit 112 does not include a rechargeable battery, but instead includes a replaceable battery or batteries, such as a pair of watch-type batteries. The battery is replaceable by removing the battery access cover 112a. The portable unit 112 having a replaceable battery obviates the need for a battery recharger in the base unit 16.

FIGS. 6a and 6b illustrate a camera 240 included as part of and for use with a monitoring system according to another embodiment of the present invention. Although not illustrated, the camera 240 may be provided with a mounting arrangement for mounting to the user's body, clothing, headwear, footwear, bicycle or other suitable location. The camera is configured for recording both video and still images which may be downloaded to a computer via a compatible portable unit and/or base unit. The camera 240 includes an indicator light 244 indicating that the camera 240 is in record mode. A button 246 is provided on the camera 240 to start and stop recording. A wind and waterproof microphone 248 is also provided to enable the user to directly record their voice onto a recording device provided in the camera 240. The camera 240 also includes a light sensor 250.

A base unit (not illustrated) is provided with male recharge members for inductively recharging the camera 240 via female members 252a, b provided on the camera. In order to charge the camera 240, the camera 240 is placed on the base unit and recharged until such time as an indicator light provided on the base unit indicates that the camera 240 has been fully charged.

To use the camera, suitable inputs are provided by the user to pair the camera 240 with an associated body unit (not illustrated). If required, the camera 240 is suitably mounted to, for example, the handle bars of a bicycle. The user can then use the camera 240 with the overall monitoring system to record video footage and/or still images while using the other features of the monitoring system. This can be of particular benefit because the user can later analyse the physiological data and/or environmental data recorded by the system in conjunction with the images provided by the camera 240 at any point during the user's exercise programme. This beneficially enables the user to be able to correlate the data provided by the system at any point in time with images at that point in time during their exercising regime to provide a better understanding as to the physiological and/or environmental data recorded.

FIGS. 7a and 7b illustrate a headset 354 for use with a monitoring system according to another embodiment of the present invention. The system may include the headset 354 in conjunction with and/or in place of the earphones 18a, b illustrated in FIG. 1. The headset 354 includes a volume up button 356 and volume down button 358. The headset 354 also includes the capability of communicating with a telephone or other communication device. A button 360 is provided on the headset 354 to indicate this mode of operation. The headset 354 is included with a comfortable rubber/silicon ear hanger 362. The headset 354 is rechargeable inductively via a suitably configured base unit (not illustrated). A music play-back button 364 is provided on the headset 354, as is a play-stop button 366 and a forward button 368. The headset 354 is waterproof.

To play music, the user presses and releases button 366. To fast forward music, the user presses and releases button 368. The fast forward mode may be configured to enable, for example, the user to press and hold button 368 for 2 seconds to go forward 10 tracks at a time.

To pair the headset 354 with a cell phone, the user presses and holds buttons 360 and 362 for 2 seconds. The user's cell phone would then prompt the user to accept the pairing. A permanent pairing can be established. To answer a call or hang up, the user would press the button 360.

The system enables the headset 354 to communicate with another paired headset. To do this, the user presses and holds buttons 356 and 360 when holding the headset 354 within, for example, 2 metres of the other headset(s) to pair with. To open a line of communication, the user would press the button 360 twice. To close a line of communication, the user would press the button 360 twice.

FIGS. 8a, 8b and 8c illustrate a miniature music receiver/communication earpiece 470 for use with a suitably configured monitoring system (not illustrated). The receiver/earpiece 470 is inductively rechargeable via the female members 472a, b provided on the receiver/earpiece 470. The receiver/earpiece 470 includes a music back button 474, a play stop button 476 and a forward button 478. A volume-up and volume-down buttons 480, 482 respectively are also provided, as is a phone/communication device and a hang-up button 483. A comfortable rubber/silicon hanger 484 is provided for mounting the receiver/earpiece to the ear of a user. An ear bud speaker 486 is provided, as is a microphone 488.

In operation, to play music through the receiver/earpiece 470, the button 476 is pressed and released. To stop music the same button is pressed and released. To fast forward music, button 478 is pressed and released to go forward one track at a time. Alternatively the button 478 can be pressed and held for 2 seconds to go forward 10 tracks at a time. The receiver/earpiece 470 may be paired with a cell phone. This is done by pressing and holding buttons 482 and 483 for 2 seconds. The user's cell phone would then prompt the user to accept the pairing. A permanent pairing can be established. To answer a call or hang up, the user presses button 483.

The monitoring system may include another or other paired headsets. To communicate between headsets, the user presses and holds buttons 480 and 483 when within, for example, 2 metres of the other headsets to be paired with. To open a line of communication between two or more headsets, the user(s) presses button 483 twice. The same button is pressed to then close the line of communication.

The present invention not only enables a user to wirelessly record physiological and environmental data, but also to be able to wirelessly listen to music while exercise. The recorded data, read from sensors integrated into the clothing of the wearer's may be later downloaded to a PC for analysis and evaluation.

The invention may be used in a wide variety of sports and other activities, including walking, cycling and running to name a few. The invention may even be provided with waterproof earphones enabling the user to listen to music while swimming.

The system may also include software for the user to better understand and monitor their training and to be able to determine increases and decreases in their fitness level. Training programs may also be provided.

The medical implications of the present invention are considerable. Sensors discreetly integrated into clothing may be used to monitor a person's heart rate for a day or over a period of many days to later be downloaded and possibly emailed to a medical practitioner for analysis and evaluation. Every heart beat of a patient could be recorded for later analysis and evaluation by the practitioner.

As discussed, the monitoring system may take various forms. For example, the system may include a portable unit, base unit, garment including various sensors and possibly software for the set-up and maximization of use of the system. Such an arrangement may be used for, for example, medical applications. The package may be used by users who need to record their body parameters for long periods of time but don't need to observe their heart rate in real time. Such an arrangement may also optionally include a heart rate monitor or other wrist mounted or bike mounted arrangement for providing a visual read-out to the user of various parameters including, for example, their heart rate. The system may further include a headset and/or earphones. One monitoring system arrangement of particular interest to professional and elite sports persons may include each of the above features, together with a system camera and an earpiece to allow the user to film their training or competition and to communicate with, for example, their trainer or coach while exercising or competing. Such a system may also include a wrist or bike mounted GPS and altitude device, whether or not these are separate or combined with the previously mentioned heart rate monitor. Still further, the system may include a plurality of portable units for enabling, for example, a coach or team manager to individually assess each of their athletes physiological levels while training and/or competing. Information from each of the portable units may be separately downloadable to a computer via one common base unit then compared and assessed.

The provision of a monitoring system having more than one portable unit and GPS capabilities enables the following scenario. Fred and John want to race their bikes around the streets. Fred races his street course at 6 am but John sleeps in and then goes out on his bike after lunch over the same course. They are then able to download the recorded data from their individual portable units and meet on line and race each other over the same course and map in real time or accelerated time. They can watch each other's body parameters, position, altitude and video whilst cheering themselves on and chatting. The map will clearly indicate if anyone took a detour from the selected course.

As another scenario for use of the monitoring system of the present invention, Bob goes for a run and finds a new running track. He saves all the information recorded from his run onto his computer. He wants to show his friend Jane the run, but Bob will be in Perth for the next week and Jane wants to run today. Bob therefore emails Jane the session and she can review the map, and also load it into her portable unit. Using the same start point, Jane can receive audio instructions to follow the route. Bob can also send coaching tips or warnings to be triggered at specific GPS locations to Jane, such as, for example, “Now I want you to sprint up this last part of the hill, go!”.

Alternatively, a route could be drawn on a map by Bob, Jane or a third party without anyone actually having previously run over that route. Bob and Jane could then run that route together or separately and later compare their physiological and/or environmental parameters recorded by their portable units.

Finally, it is to be understood that various alterations, modifications and/or additions may be introduced into the construction and arrangement of the parts previously described without departing from the spirit or ambit of this invention.

Without limiting the claim scope that may be sought in any Australian or overseas patent application claiming priority from the present application, the following claims are provided.

1. A system for monitoring physiological parameters of a user, the system comprising:
(a) a sensor unit comprising:
a sensor for sensing physiological information for at least one physiological parameter of the user; and
a transmitter for wirelessly transmitting a first signal containing the sensed information;
(b) a portable unit comprising:
a portable unit controller;
a memory for storing preprogrammed information;
a receiver for receiving, under the control of the controller, the first signal into memory as sensed physiological data;
a transmitter for wirelessly transmitting, under the control of the controller, a second signal containing the preprogrammed information; and
(c) a user output device comprising a second receiver for receiving the second signal and a converter for converting the second signal into an output that is audible to the user, and wherein
the sensor unit further comprises a sensor for sensing environmental information for at least one environmental parameter to which the user is exposed.

2. A monitoring system according to claim 1, wherein the memory contains preprogrammed information.

3. A monitoring system according to claim 2, wherein the preprogrammed information is provided in at least one data file.

4. A monitoring system according to claim 2, wherein the preprogrammed information comprises audio information.

5. A monitoring system according to claim 4, wherein the audio information is provided in at least one of an mp3 file, an mp4 file, an m4a file or other current audio format.

6. A monitoring system according to claim 1, wherein the second signal transmitted by the transmitter of the portable unit comprises both the preprogrammed information and information related to the sensed physiological data.

7. A monitoring system according to claim 1, wherein the transmitter of the sensor unit and the receiver of the portable unit are connected by a Bluetooth connection and the first signal is a Bluetooth signal, and
wherein the transmitter of the portable unit and the receiver of the user output device are connected by a Bluetooth connection and the second signal is a Bluetooth signal.

8. (canceled)

9. A monitoring system according to claim 1, wherein the portable unit is worn or carried by the user, and
wherein the sensor of the sensor unit is integrated into fabric or material of an article to be worn by the user.

10. (canceled)

11. A monitoring device according to claim 1, wherein the sensor unit comprises a plurality of sensors for sensing a variety of different physiological parameters, and
wherein the physiological parameters comprise body temperature, skin temperature and heart rate.

12. (canceled)

13. A monitoring system according to claim 1, wherein the controller comprises a processing unit and software for controlling the composition and wireless communication of the second signal from the transmitter of the portable unit to the receiver of the user output device.

14. A monitoring system according to claim 1, further comprising a base unit, the base unit comprising a docking facility for docking the portable unit thereto, and comprising:
a base unit transmitter for wirelessly transmitting a third signal containing the preprogrammed information to a second receiver of the portable unit for storage in the memory of the portable unit, and
a base unit receiver for receiving a fourth signal containing the sensed physiological data from the memory of the portable unit by way of a second transmitter of the portable unit.

15. (canceled)

16. A monitoring system according to claim 15, wherein
the transmitter of the base unit and the second receiver of the portable unit are connected by a Bluetooth connection and the third signal is a Bluetooth signal,
the receiver of the base unit and the second transmitter of the portable unit are connected by a Bluetooth connection and the fourth signal is a Bluetooth signal,
the receiver and second receiver of the portable unit are integrated, and
the transmitter and second transmitter of the portable unit are integrated.

17-19. (canceled)

20. A monitoring system according to claim 1, wherein the at least one environmental parameter comprises at least one of the user's location, altitude, ambient air temperature, humidity and barometric pressure.

21. A monitoring system according to claim 1, wherein the sensor for sensing the environmental information is integrated into the portable unit.

22. A monitoring system according to claim 1, wherein the user output device comprises at least one of a pair of wireless earphones, a pair of wireless headphones, a wireless headset and a wireless communication earpiece, and further comprises an audio output controller.

23. (canceled)

24. A monitoring system according to claim 1, further comprising a camera for recording images while using the monitoring system,
wherein at least one of the portable unit user output device and camera comprises a rechargeable battery and the base unit comprises a battery recharger for recharging the at least one battery, and
wherein the battery charger is an inductive battery charger.

25-26. (canceled)

27. A monitoring system according to claim 1, further comprising a portable monitor having a display for displaying information relating at least one of the physiological data, environmental data and preprogrammed information.

28. A monitoring system according to claim 1, wherein the portable unit is integrated into the user output device, and wherein the sensor unit comprises at least one sensor for sensing at least one of the acceleration and inclination.

29. (canceled)

30. A system for monitoring physiological parameters of a user, the system comprising:
(a) a sensor unit comprising:
a sensor for sensing physiological information for at least one physiological parameter of the user; and
a transmitter for wirelessly transmitting a first signal containing the sensed information;
(b) a portable unit comprising:
a portable unit controller
a memory for storing preprogrammed information;
a receiver for receiving, under the control of the controller, the first signal into memory as sensed physiological data;
a transmitter for wirelessly transmitting, under the control of the controller, a second signal containing the preprogrammed information; and wherein
the sensor unit further comprises a sensor for sensing environmental information for at least one environmental parameter to which the user is exposed.

31. A system for monitoring physiological parameters of a user according to claim 30, further comprising a base unit, the base unit comprising a docking facility for docking the portable unit thereto.

32. (canceled)
